(19) United States
(12) Patent Application Publication
(10) Pub. No.: US 2013/0116570 A1
(43) Pub. Date: May 9, 2013

(54) LOCAL COMPRESSION DURING AUTOMATED ULTRASOUND SCANNING AND METHODS OF ACOUSTIC COUPLING

(71) Applicant: The Regents Of The University Of Michigan, Ann Arbor, MI (US)

(72) Inventors: Paul L. Carson, Ann Arbor, MI (US); Gerald L. Lecarpentier, Plymouth, MI (US); Christine R. Lashbrook, Whitmore Lake, MI (US); Won-Mean Lee, Ann Arbor, MI (US); Mitchell M. Goodsitt, Ann Arbor, MI (US); Kazuhiro Saitou, Ann Arbor, MI (US); Boyun Wang, Ann Arbor, MI (US); Seunghun Baek, Ann Arbor, MI (US); J. Brian Fowlkes, Ann Arbor, MI (US)

(73) Assignee: The Regents Of The University Of Michigan, Ann Arbor, MI (US)

(21) Appl. No.: 13/655,962

(22) Filed: Oct. 19, 2012

Related U.S. Application Data
(60) Provisional application No. 61/549,352, filed on Oct. 20, 2011.

(51) Int. Cl. A61B 8/00

(52) U.S. Cl. CPC A61B 8/4281; A61B 8/403; A61B 8/4416
USPC 600/459

(57) ABSTRACT

A system for imaging a portion of a body, such as a human breast. The system comprises a first scanning system that ultrasonically scans a portion of a body while a compression system applies a compressing force to the portion of the body being scanned. A device is used for containing an acoustic coupling gel for enhanced acoustic coupling with the portion of body.

### CROSS-REFERENCE TO RELATED APPLICATIONS

This application claims the benefit of U.S. Provisional Application No. 61/549,352, filed on Oct. 20, 2011. The entire disclosure of the above application is incorporated herein by reference.

### GOVERNMENT INTEREST

This invention was made with government support under Grant No. CA091713 awarded by the National Institutes of Health. The government has certain rights in the invention.

### FIELD

The present disclosure relates to ultrasound imaging and, more particularly, to local compression during automated ultrasound scanning and method of acoustic coupling to the breast.

### BACKGROUND & SUMMARY

This section provides background information related to the present disclosure which is not necessarily prior art. This section also provides a general summary of the disclosure, and is not a comprehensive disclosure of its full scope or all of its features.

Breast cancer is the second leading cause of cancer deaths in women today. About 1.3 million women are diagnosed annually worldwide and about 465,000 will die from the disease. Mammography (breast imaging) is an important early diagnostic tool for the detection of breast cancer and other medical conditions effecting women's health. Mammography techniques have continually improved since their inception.

Automated ultrasound (AUS) imaging is a very promising breast cancer screening technology that can detect cancers in dense breasts, particularly with extreme compounding. However, automated ultrasound scanning (AUS) of the breast has developed more slowly than anticipated. The main limitation, beyond achieving adequate acoustic coupling to the breast, has been excessive shadow artifacts. Shadow artifacts occur when reflecting structures at acute angles to the ultrasound beam are not flattened by the transducer as well as in manual scanning. It is believed that imaging of the breast in near mammographic compression provides much of the needed flattening to overcome these limitations.

According to the teachings of the present invention, it has been found that during breast AUS under very light mammographic compression, local compression by the transducer can be used to flatten the acutely oriented structures further and reduce the acoustic path length to key structures in the breast. In doing so, it is now possible to scan the breast in the same system, and in some embodiments, nearly simultaneously, as mammography or digital breast tomosynthesis (DBT).

By way of the present teachings, it is shown that artifacts can be significantly reduced in AUS using a very light mammographic compression by a flexible mesh in a frame, along with additional localized compression of the breast as the transducer is scanned in contact with the mesh and the breast. The system of the present teachings revolutionizes automated breast cancer screening and greatly accelerates the tempo of scientific research on and practice in breast imaging.

The present teachings further aid in the application and containment of coupling gels, lotions, or other viscous materials around the breast during screening, which often necessary for sufficient ultrasound image quality. These gels, lotions, or other viscous materials serve, in part, to exclude air from any acoustic path aimed at breast tissue. The present teachings are particularly well suited for imaging the breast compressed in the mammographic geometry and may also be used for automated ultrasound imaging in the currently more common supine geometry. In the embodiments, the principles of the present teachings enable a single image volume sweep of the entire breast rather than 2-5 sweeps of similar length to cover the breast.

Further areas of applicability will become apparent from the description provided herein. The description and specific examples in this summary are intended for purposes of illustration only and are not intended to limit the scope of the present disclosure.

### DRAWINGS

The drawings described herein are for illustrative purposes only of selected embodiments and not all possible implementations, and are not intended to limit the scope of the present disclosure.

FIG. 1 is a perspective view illustrating the dual-modality imaging system according to the principles of the present teachings;

FIG. 2 is a top perspective view illustrating the dual-modality imaging system according to the principles of the present teachings;

FIG. 3 is a side perspective view illustrating the dual-modality imaging system according to the principles of the present teachings;

FIG. 4 is a side view illustrating the compression frame assembly according to the principles of the present teachings;

FIG. 5 is a perspective view illustrating the compression frame assembly employing a gel dam according to the principles of the present teachings;

FIG. 6 is a schematic of the system according to some embodiments of the present teachings;

FIG. 7 is a schematic of a transducer assembly according to some embodiments of the present teachings;

FIG. 8 is a photograph of a mesh bra for use with the system of the present teachings;

FIG. 9 is a photograph of a mesh bra for use with the system of the present teachings;

FIG. 10 is a photograph of a mesh bra for use with the system of the present teachings with a phantom breast contained therein;

FIG. 11 is a perspective view illustrating the compression frame assembly employing a gel dam according to the principles of the present teachings;

FIG. 12 is a top view illustrating a gel dam according to the principles of the present teachings;

FIG. 13 is a schematic diagram of the system of the present teachings;

FIG. 14 are graphs illustrating force profiles during examination of a patient;

FIG. 15 illustrates a graphical user interface of the present teachings;

FIG. 16 is a photograph of a polyester cloth gel roll according to the principles of the present teachings;

FIG. 17 is a schematic of a dual partitioned gel roll according to the principles of the present teachings;

FIG. 18 is a schematic of a belt retained, gel roll according to the principles of the present teachings;

FIG. 19 is a schematic of a belt retained, gel roll using wedges according to the principles of the present teachings;

FIG. 20 is a schematic of a plurality of actuators applying the gel roll to the breast according to the principles of the present teachings;

FIG. 21 is a schematic of a plurality of actuators applying the gel roll to the breast according to the principles of the present teachings;

FIG. 22 is a schematic of a conveyor retention system for supporting a breast according to the principles of the present teachings;

FIG. 23 is a schematic of a tilt examination system according to the principles of the present teachings; and

FIG. 24 is a schematic of a rapid gel distribution system according to the principles of the present teachings.

### Corresponding reference numerals indicate corresponding parts throughout the several views of the drawings.

### DETAILED DESCRIPTION

Example embodiments will now be described more fully with reference to the accompanying drawings. Example embodiments are provided so that this disclosure will be thorough, and will fully convey the scope to those who are skilled in the art. Numerous specific details are set forth such as examples of specific components, devices, and methods, to provide a thorough understanding of embodiments of the present disclosure. It will be apparent to those skilled in the art that specific details need not be employed, that example embodiments may be embodied in many different forms and that neither should be construed to limit the scope of the disclosure.

The terminology used herein is for the purpose of describing particular example embodiments only and is not intended to be limiting. As used herein, the singular forms “a”, “an” and “the” may be intended to include the plural forms as well, unless the context clearly indicates otherwise. The terms “comprises,” “comprising,” “including,” and “having,” are inclusive and therefore specify the presence of stated features, integers, steps, operations, elements, and/or components, but do not preclude the presence or addition of one or more other features, integers, steps, operations, elements, components, and/or groups thereof. The method steps, processes, and operations described herein are not to be construed as necessarily requiring their performance in the particular order discussed or illustrated, unless specifically identified as an order of performance. It is also to be understood that additional or alternative steps may be employed.

When an element or layer is referred to as being “on”, “engaged to”, “connected to” or “coupled to” another element or layer, it may be directly on, engaged, connected or coupled to the other element or layer, or intervening elements or layers may be present. In contrast, when an element is referred to as being “directly on,” “directly engaged to”, “directly connected to” or “directly coupled to” another element or layer, there may be no intervening elements or layers present. Other words used to describe the relationship between elements should be interpreted in a like fashion (e.g., “between” versus “directly between,” “adjacent” versus “directly adjacent,” etc.). As used herein, the term “and/or” includes any and all combinations of one or more of the associated listed items.

Spatially relative terms, such as “inner,” “outer,” “beneath”, “below”, “lower”, “above”, “upper” and the like, may be used herein for ease of description to describe one element or feature's relationship to another element(s) or feature(s) as illustrated in the figures. Spatially relative terms may be intended to encompass different orientations of the device in use or operation in addition to the orientation depicted in the figures. For example, if the device in the figures is turned over, elements described as “below” or “beneath” other elements or features would then be oriented “above” the other elements or features. Thus, the example term “below” can encompass both an orientation of above and below. The device may be otherwise oriented (rotated 90 degrees or at other orientations) and the spatially relative descriptors used herein interpreted accordingly.

### Introduction

According to the principles of the present teachings, an apparatus and method is presented that make it possible and practical to image a large fraction (˜90%) of the breast with automated 3D ultrasound at typically high ultrasound frequencies. The apparatus and methods of the present teachings are capable of achieving the aforementioned with the same or slightly reduced compression as an accompanying mammogram or digital breast tomosynthesis (DBT) volumetric image set. The same geometry insures one-to-one correspondence between the lesions observed in the X-ray and ultrasound images. Because ultrasound and mammography or DBT show such different properties of breast abnormalities, more cancers can be detected if 3D ultrasound studies are performed. This nearly orthogonal information also means that if 3D ultrasound and DBT are performed in the same views, many possible lesions seen in only one modality can be eliminated as possible cancers, reducing the high rates of call backs from screening for further imaging or biopsy.

In the past, other efforts to perform automated ultrasound in the mammographic geometry failed because:

1) The breast slipped out partially from between the compression paddles when gels were applied to the breast in order to eliminate air between the ultrasound transducer and the breast, i.e., in order to provide acoustic coupling;

2) Solid compression paddles were employed that supported ultrasound reverberations, absorbed the ultrasound, and otherwise distorted the images;

3) The compression paddles were flat, rather than allowing an edge of the paddle frame at the chest wall to compress the retromammary fat and allow the breast to bulge up beyond the frame to hold the breast in place;

4) Air was trapped between the breast and the compression paddle, particularly where the breast began to curve away from the paddle;

5) Gel did not extend out beyond the edges of the breast well enough to assure that all of the scanning ultrasound transducer elements were always sending and receiving sound through bubble free gel to the breast;

6) Only part of the full thickness of the breast could be imaged with the high frequencies usually employed to see weak tissue scatterers and to image with high resolution;

7) Application of the gel after the DBT or mammogram allowed for some motion between the X-ray and ultrasound and made gel application more difficult with the breast already in place; and

8) The Transducer could not get as close to all objects in the breast when the breast was flattened as it could if the breast were compressed primarily where the narrow transducer was scanning at the time.

Those failures, by number, are overcome by the apparatus and method of the present teachings by: (1-4) using a cloth mesh stretched across a thin, rigid frame. The mesh is not made as slick by the ultrasound coupling gels as is a solid surface (1). The thin mesh chosen does not distort the ultrasound nearly as much as does a solid paddle (2). The mesh is stretched up by the breast inside the frame, trapping the breast (3). The mesh allows gel to pass through, so gel on the breast oozes through the mesh, preventing trapping of gas and providing contact of the transducer with the gel. The present teachings further use very viscous gels to allow large areas of gel to support themselves without oozing through the mesh or flowing away too rapidly (5). In some embodiments, a gel dam is used to hold the gel in place around the breast without the gel flowing away from the breast. In some embodiments, the gel can be further constrained by being in a bra, a mesh tube, and/or being a solid gel. According to some embodiments, gel application devices can be used to apply large amounts of gel around the breast rapidly, with minimal trapped air bubbles and minimal mess. To overcome the disadvantage of (6) above, the present teachings can employ ultrasound transducers and mesh paddles on both sides of the breast. This makes solution of failure 5 more difficult and critical, having two sides of the breast to keep in acoustic contact with the transducers. In some embodiments, use of a gel/emulsion that attenuates the X-rays less than water can be used to overcome failure (7). In some embodiments, methods are provided that reduce the tension on the mesh so that the mesh can be compressed more by at least one of the transducers as they scan over the breast to overcome failure (8). Controlling the height of the transducer as it scans over the breast provides local, rather than global, compression.

Each of the aforementioned features will be more directly described herein.

Ultrasound imaging of the breast provides complementary information to conventional X-ray mammography. A primary example is the well-established use of ultrasound in differentiating solid masses from cysts. Another contribution of ultrasound in breast imaging is its utility in characterizing solid masses themselves. In fact some studies, using strictly enforced evaluation criteria, indicate that current ultrasound technology may be a reliable means of identifying solid masses as benign or malignant and may be especially useful in detecting and diagnosing mammographically occult masses.

Despite these benefits, however, conventional ultrasound imaging is typically performed freehand in a geometry different from that of mammography. This may result in difficulties correlating areas of interest in the two image modalities. In addition, conventional breast ultrasound scanning is highly operator-dependent and requires skillful probe manipulation and the mental ability of the operator to envision 3-dimensional (3D) tissue structure. Accurate diagnoses using breast sonography alone is also problematic as indicated by numerous studies showing high false-positive and false-negative rates.

The X-ray/ultrasound mammographic system 10 of the present teachings can address these problems by combining a digital X-ray of the compressed breast with a subsequent 3D ultrasound scan in the same orientation (breast compression being relaxed just enough for patient comfort). Such a system produces X-ray and ultrasound images in the same conventional mammographic imaging geometry. These combined images could be helpful in the assessment of suspicious regions, given that simultaneous identification of multiple features suggestive of malignancy leads to higher diagnostic confidence.

It has been reported that screening with both X-ray and ultrasound imaging modalities performed by a skilled physician using high quality equipment provides high correlation between ultrasound and X-ray mammography inexpensively and improves cancer detection significantly.

Dual-mode whole-breast imaging also exhibits significant potential for advanced modes that can provide additional information about breast tissue not available using conventional mammography and ultrasound imaging. In particular, X-ray DBT could replace digital mammography in a combined system for 3D co-delineation of tissue structures. Even fusion of conventional digital mammography images with co-registered ultrasound exams greatly reduces ambiguity in correlation of findings in the two exams. Initial results of such fusion of pulse echo ABU and of digital mammography or of DBT are promising, although there remain technical issues of breast coverage and some acoustic coupling artifacts in ABU.

According to the principles of the present teachings, improved automated breast imaging has been developed, including a standalone and dual-modality system, both in the mammographic geometry. These systems have an unprecedented impact on the accuracy of breast cancer screening, diagnosis and treatment planning, and assessment. The main effect has been the earlier diagnosis by at least one year of over 40% of cancers in dense breasts.

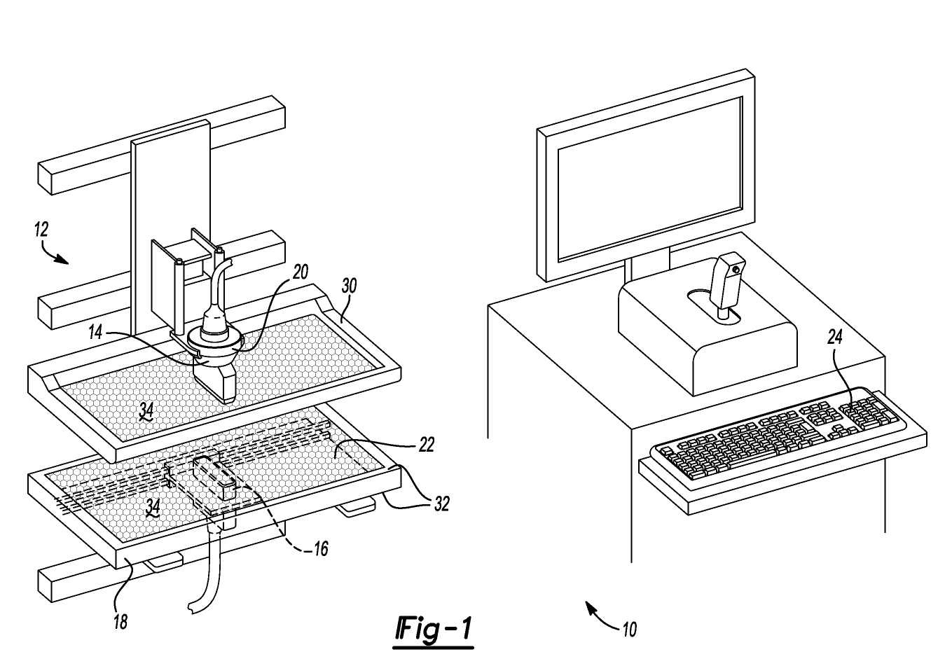

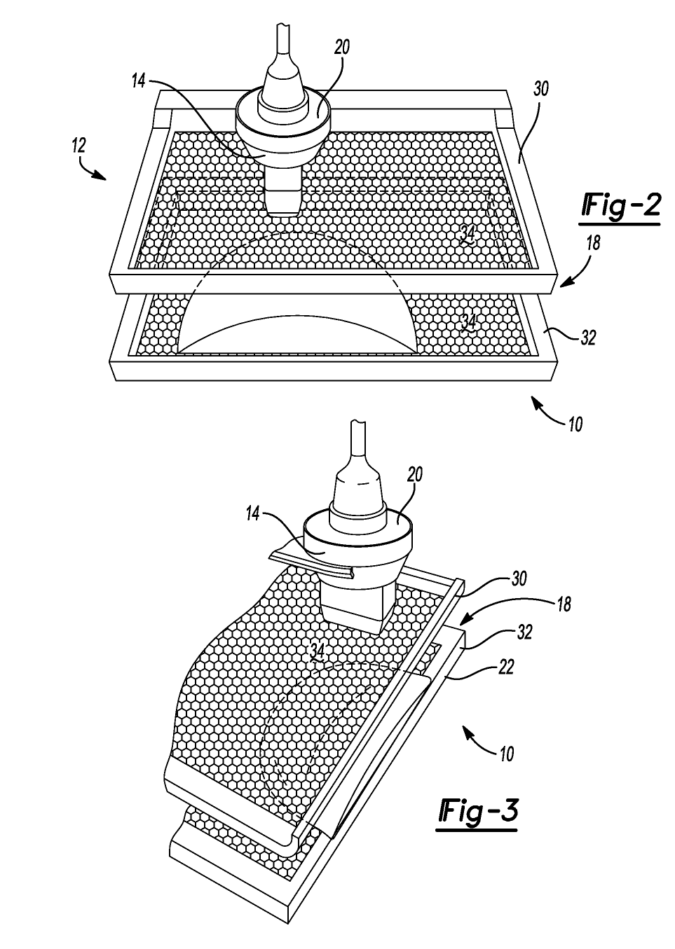

With particular reference to FIGS. 1-7, a system for imaging a human breast is illustrated according to the principles of the present teachings. The system 10 can be used for dual-modality imaging, including the combination of conventional X-ray imaging (or digital breast tomosynthesis imaging) and ultrasonic imaging. Generally, the system 10 comprises a conventional X-ray imaging system (or digital breast tomosynthesis imaging system) and a support structure 12 supporting a plurality of components. The support structure 12 can comprise an upper or first transducer assembly 14, a lower or second transducer assembly 16, a compression frame assembly 18, a upper or first transducer drive assembly 20, a lower or second transducer drive assembly 22, and a control system 24 operably coupled to upper transducer assembly 14, lower transducer assembly 16, upper transducer drive assembly 20, and lower transducer drive assembly 22. It should be appreciated that variations of the present teachings are envisioned and anticipated thereby. It should also be appreciated that some components, such as, but not limited to, lower transducer assembly 16 and lower transducer drive assembly 22 may be optional in some embodiments.

The present teachings provide an imaging system that is particularly well suited for imaging a patient's breast while in an upright position, which is also known generally as a mammographic geometry. However, it should be understood that the principles of the present teachings are also useful in other patient orientations, including a supine geometry. Moreover, it should be further understood that although the present description is provided in connection with breast imaging, the apparatus and methods described herein can be used for imaging any human body part, with obvious modification to structure.

With continued reference to FIGS. 1-7, compression frame assembly 18, in some embodiments, can comprise an upper frame member or compression paddle 30 and an opposing lower frame member or compression paddle 32. Upper frame member 30 and lower frame member 32 can each comprise a generally rectangular metallic structure defining an open interior operable to permit ultrasound and other imaging signals therethrough. In some embodiments, as described more completely herein, each of upper frame member 30 and lower frame member 32 can be covered in a mesh 34 that permits imaging of the patient's breast. Mesh 34 is also chosen to aid in the containment of gel 36 used for aiding acoustic coupling between the transducers of upper transducer assembly 14 and lower transducer assembly 16 and the patient's breast tissue. Mesh 34 replaces the standard plastic mammography compression paddle. The transducer, in contact with the mesh and the breast, can be physically translated by upper transducer drive assembly 20 and lower transducer drive assembly 22.

Breast phantoms and the breasts of three women were scanned with usual compression by the mesh paddle and then with less global, but added local, compression.

As will be discussed herein, the system 10 is particular well suited for imaging a patient's breast via two-sided ultrasound imaging (e.g. upper and lower). The patient's breast is lightly compressed through the application of a global or overall compressive force applied by compression frame assembly 18. However, system 10 is further capable of applying local compression to the patient's breast through the direct application of the transducers of upper transducer assembly 14 and lower transducer assembly 16. This local compression can be manually controlled by an operator and/or automatically controlled by system 10. Coupling gel 36 can be used to improve acoustic coupling. Coupling gel 36 can be easily applied to the patient's breast prior to insertion into system 10. Moreover, due to the nature of mesh 34, gel 36 is permitted to permeate mesh 34 to allow gel 36 to establish improved acoustic coupling of opposing sides of mesh 34. As will be discussed, according to some embodiments of the present teachings, gel barriers or dams (FIG. 5) can be used to contain gel 36 between upper frame member 30 and lower frame member 32 of compression frame assembly 18. Moreover, in some embodiments, mesh bras can further be used for gel containment relative to the patient's breast.

### Compression

Automated ultrasound imaging is traditionally limited in resolution and contrast due to increased artifacts from the tissues themselves compared with what is achieved with hand control of the transducer array. Mammographic compression does eliminate some artifacts in that aberrating internal tissue borders are slightly flattened and the beam has shorter distances to travel to internal structures. It has been determined that the ultrasonic breast imaging effort at Bath, UK, observed excessive attenuation artifacts in their different geometry that were eliminated by manual compression with a transducer. It is believed that there is much to be gained in breast scanning geometry by very light mammographic compression by a flexible material in a frame, along with local additional compression of the breast as the transducer is scanned in contact with the material and the breast.

Reasonable distortion of the breast by the local compression can be corrected if necessary by image registration to the 3D X-ray image, Mammographic Tomosynthesis (MT), or by an ultrasound scan performed exactly in the same compression as the X-rays. The concept of local compression in the mammographic geometry for relatively precise correlation between the two imaging modalities is new and is a breakthrough that makes screening by ultrasound plus X-rays a reality in the United States, as well as much of the world.

According to the principles of the present teachings, comprssion can be applied by at least application via a biasing member (e.g. spring), manual control by an operator, or remote or automatic control of the local compression force or displacement. If a remote control device by an operator is used, tactile and optical and possibly other feedback can be provided on the compression force as well as resistance to motion of the compressing object by the breast. That is, compression control can be performed while computer controlled motors or electronic switching controls the location of a compression device, such as the ultrasound transducer itself.

Remotely controlled local compression of regular scanning can be applied in imaging and/or therapy of the breast and other body parts. The approach can produce more uniform scanning and be more ergonomic for the operator and can lead to automated scanning with minimal operator intervention. Four human studies of the local compression concept have been performed in this geometry with good effect. Software is involved but not primary.

To provide further improved techniques, initial imaging tests were performed in a combined AUS/DBT system. Visual indicators of image features expected to provide improved medical imaging were observed with local compression. Also, lateral movement of tissues appeared acceptably small. These results motivated design and construction of an apparatus to make local compression practical and safe. Examples of flattened structures were observed more brightly in the locally compressed breasts, and acoustic paths longer than 35 mm were reduced by ˜10 mm. In many areas, image penetration was 3 cm or greater. In one case, image volumes with or without local compression were spatially aligned by nonlinear image registration software.

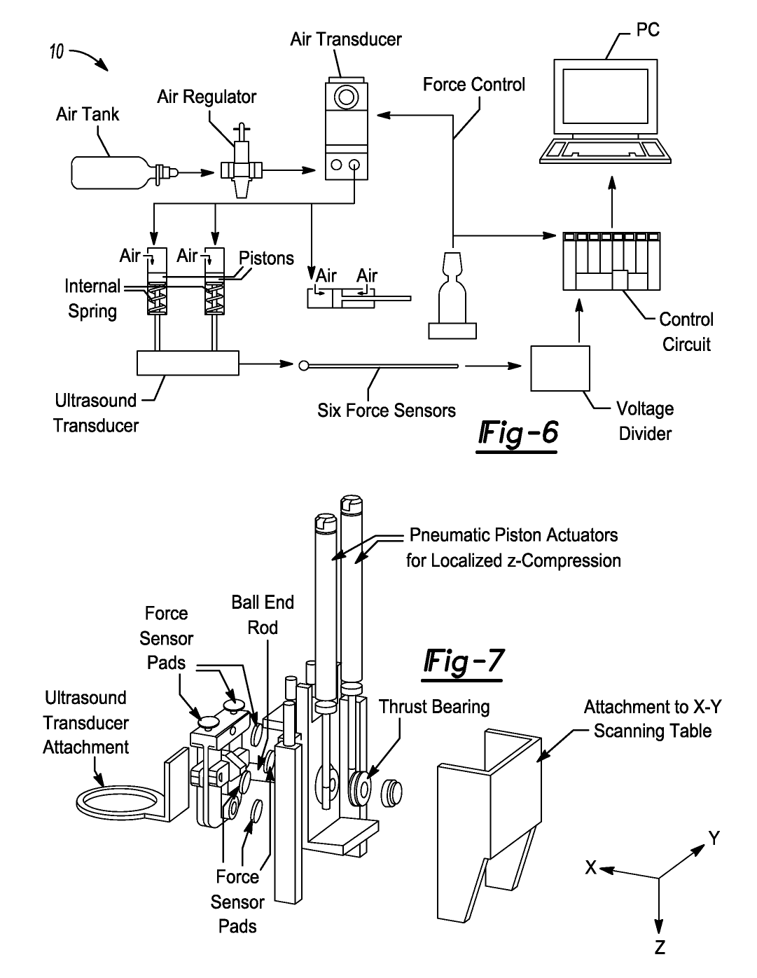

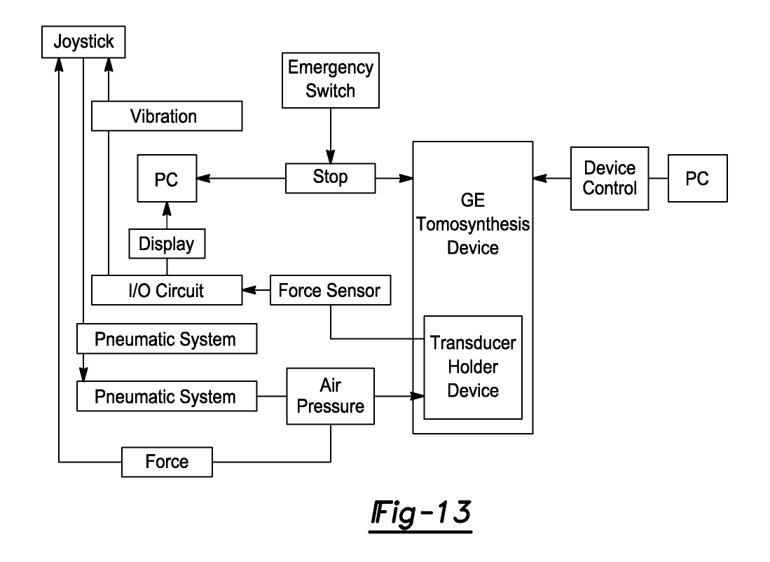

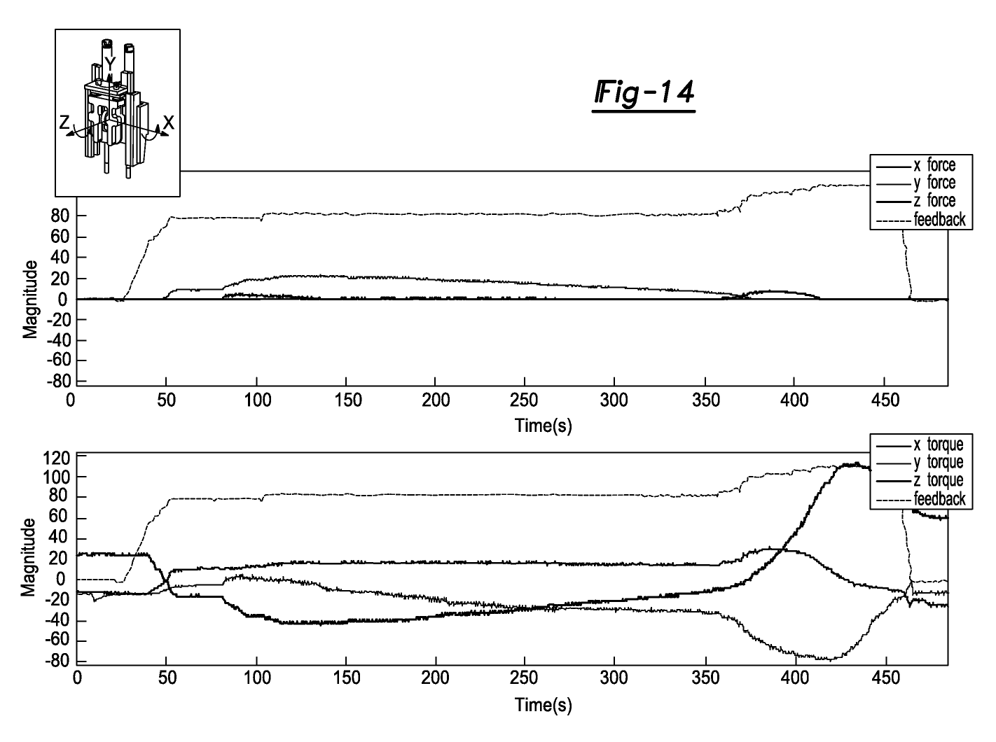

In some embodiments, the designed system of the present teachings entails operator control of vertical compression force by an ultrasound transducer array during the localized compression scanning (FIG. 6). The operator is provided with feedback of the vertical compression force on the breast exerted by the ultrasound transducer, and with vibrotactile feedbacks of the reaction torques from the breast on the ultrasound transducer. The vertical compression of the breast is realized by pneumatic actuators attached to the transducer controlled by the remote operator with a joystick. A schematic illustration of such system is provided in FIG. 13. The joystick is pneumatically actuated with the same air pressure as that applied to the actuators for breast compression, providing the force feedback of the compression force to the operator. This allows robust and cost-effective force feedback of the compression force without the need of additional sensors and actuators.

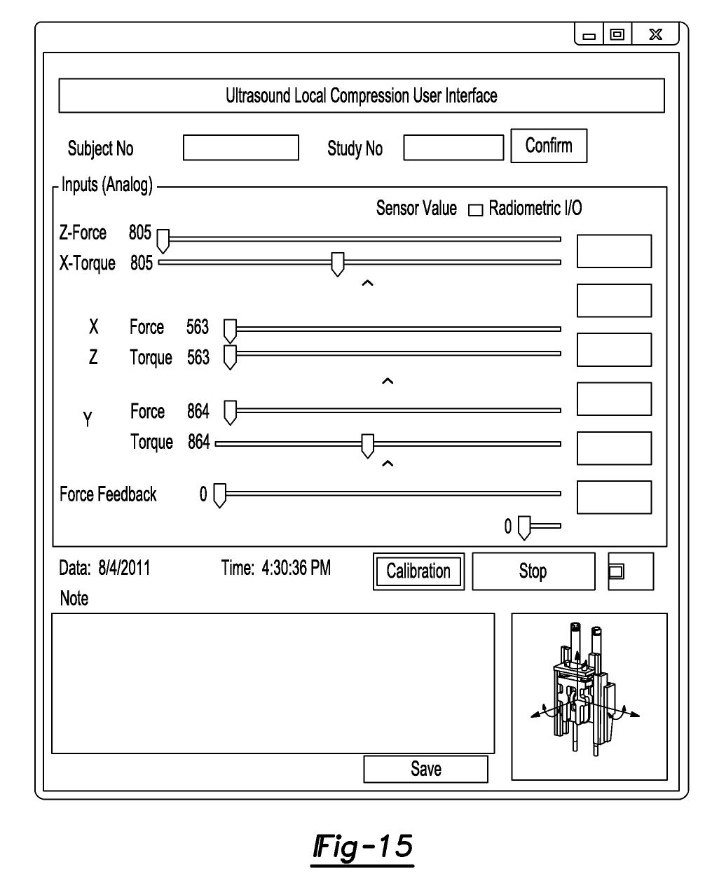

In some embodiments, the ultrasound transducer is attached to the pneumatic actuators with a custom holder equipped with 6 force sensors whose measurements can be used to compute the two-axis reaction torques on the transducer (FIG. 7). Two miniature vibrator pads attached to the joystick provide vibrotactile feedbacks to the operator proportional to the magnitudes of the computed reaction torques. Availability of the vibrotactile feedbacks of the two reaction torques are intended to effectively reduce the potential risk of injury if the transducer does not slide over stiff breast structures, with minimum additional cost and disturbances to the patients. A graph of force measured forces is provided in FIG. 14 and the associated graphical user interface in FIG. 15.

During localized compression scanning process, a computer can record the measured forces as well as the air pressure in the pneumatic system and adapts the feedback gains to individual operators and patients within set limits. The system is designed to be fail-safe such that no compression force is applied to the breast in case of power loss or the emergency shut-down by the operator.

### Compression Paddles

Traditional mammography compression paddles cannot be used for combined X-ray and ultrasound imaging because the paddles are made out of a plastic material (usually polycarbonate) that is too absorbing of ultrasound. Even paddles made of special plastics that are much less ultrasound absorbing, such as TPX, are not ideal for this application because of the need to use ultrasound coupling material on both sides of the paddle (i.e., between the ultrasound transducer and the paddle and between the paddle and the breast), This requirement increases the odds of having poor acoustic coupling at some points in the scan which degrades ultrasound image quality.

A better alternative is to use a compression paddle made out of a mesh material with pores that are large enough to permit ultrasound coupling media (gel) to pass through the mesh 34.

According to the initial teachings, a “mesh” paddle (aka compression frame assembly 18 and mesh 34) was developed that offers distinct advantages for certain procedures including a needle biopsy wire localization. In some embodiments, the paddle consists of a rigid aluminum frame with a multitude of holes through which special fish line is strung. Unfortunately, it was determined that such paddle design has certain drawbacks that limit its usefulness in the patient environment. First, this strung paddle design requires stringing much like a tennis racket, and in the event of failure of the fish line “string” or contamination of the string, it would need to be restrung, which would be inconvenient, time consuming and costly.

According to another embodiment of the present teachings, a compression mesh paddle design was developed that incorporates the advantages of the previous design while solving the problems listed above. In order to improve the imaging aspects of the paddle, the string of the initial design was replaced with a pre-formed mesh material. This pre-made mesh eliminates the artifacts generated in the ultrasound imaging. The mesh also eliminates the extensive time required to string the paddle. Selection of the mesh material will be discussed below.

### Acoustic Coupling Using Gels

### Mesh Bras

In some embodiments, acoustic coupling gels can be used to more fully couple the ultrasound transducer with the patient's breast. A viscous gel, lotion, or other viscous material that will not drip from reasonable masses of gel at or near body temperature is required for acoustic coupling in ultrasound imaging of the breast in the mammographic geometry. In some embodiments, such as illustrated in FIGS. 8-10, large amounts of bubble free gel can be placed in an acoustically transparent mesh bra. The mesh bra can then be placed on the patient and then place the bra, gel, and breast can be within compression frame assembly 18 prior to the application of global compression.

### Gel Dam

In some embodiments, as illustrated in FIG. 5, gel can be applied and/or contained using a dam or barrier. The gel dam can be used to keep the large amounts of gel from spreading and dripping on the patient and equipment, which can also lead to the introduction of air or air bubbles along the acoustic transmission path.

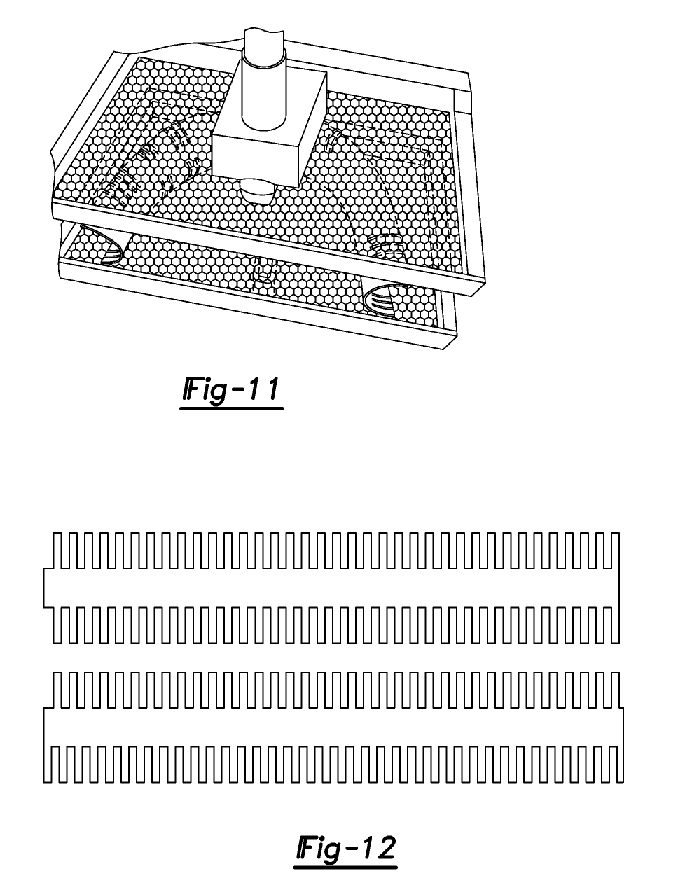

In some embodiments, as illustrated in FIGS. 11 and 12, the gel dam can comprise a thin strip of rubber. Rubber is selected because of its well matched acoustic impedance relative to the gel and its high attenuation of ultrasound, which minimizes acoustic reflections. The rubber strip can define a plurality of slits along its longitudinal sides to allow bending of the sheet in two dimensions. A strip of rubber is chosen for the approximately correct dimensions of the patient's breasts.

During use, the gel dam is placed between the compression frame assembly 18 and compressed slightly while shaping the strip to bend convexly toward the patient. The gel dam is then shaped into the shape of the breast but removed by ˜2 cm from the expected closest approach of the compressed breast. The strip should be placed to contact the chest wall on either side of the breast, particularly the inferior side of the breast in lateral and MLO views to keep the gel from slowly flowing down under gravity. Large amounts of gel are applied on the patient side of the curved strip, the breast placed between the paddles, and then compressed by the maximum comfortable amount. Additional gel may be applied from above or below the breast through the mesh paddles and for coupling to the transducers. This may also be done with the breast contained in a gel bra, but probably a more anatomically molded, seamless bra of mesh material than would be required without the containment dam.

### Mesh Selection

One complication associated with performing ultrasound scans through the conventional polycarbonate compression paddle in a combined X-ray/ultrasound breast imaging system is the degradation of the ultrasound image volume resulting from the absorption and reflection of the ultrasound beam by the paddle. Additional challenges are (1) the need to continuously stabilize the breast between the compression paddle and X-ray detector for both X-ray and ultrasound scans and (2) the need to maximize breast coverage by the ultrasound imaging system. Thus, ease of use as well as effective and time-economic acoustic coupling of the breast to the compression paddle and compression paddle to the ultrasound transducer are essential. This double acoustic coupling requirement for a solid compression paddle is associated with a greater probability of coupling (e.g., air bubble) artifacts than in hand-controlled contact scanning. Also, it is difficult to achieve adequate coupling at the breast periphery, where the breast curves away from the solid paddle. A promising alternative compression paddle is one made of mesh that is held taught within a thin rigid frame. If the mesh pores are sufficiently large, ultrasound gel can pass through the pores and provide acoustic coupling to the breast both where the mesh is in direct contact with the breast and where the mesh is several cm in distance from the skin.

The mesh candidates for compression frame assembly 18 that were considered are generally thin enough and of low enough atomic number and density that they are not visible on the breast X-ray images; however, they do display different acoustic properties. Preliminary subjective studies, with polypropylene and polyester surgical mesh samples from Textile Developments Associates, Inc. (Brookfield, Conn.) indicated that several polyester mesh materials were promising with low acoustic attenuation and minimal artifacts. Other meshes, including most polypropylene and some polyester, with different weave patterns, material thicknesses, and pore sizes exhibited significant attenuation and in some cases, significant artifacts.

Accordingly, the following represents the results of an attempt to objectively quantify the acoustic effects of the promising polyester mesh samples as well as a paddle employing an ultrahigh molecular weight polyethylene fish line (Dyneema) tightly strung across a frame, much like a tennis racquet. Also included in this analysis was a solid Polymethylpentene (TPX) plastic compression paddle that was employed in most of our initial dual-modality studies. The TPX material has the lowest density of any thermoplastic, giving it an ultrasonic impedance of 1.7 RayI, and an attenuation of 5 dB/cm at 5 MHz. Since it is an entirely aliphatic polymer, its X-ray attenuation coefficient is also small.

### Materals and Methods

### Experimental Conditions

The effects of various mesh samples on ultrasound image quality were evaluated by imaging a Computerized Imaging Reference Systems (CIRS) model 047 gray scale contrast detail ultrasound phantom (Computerized Imaging Reference Systems, Inc., Norfolk, Va.). This phantom is made of Zerdine (attenuation coefficient equal to 0.50±0.05 dB/cm-MHz, sound speed of 1540 m/s±10 m/s) and contains test cylinders of 2.4, 4, and 6.4 mm diameters, each tilted downward. The objects of highest contrast were imaged over a broad range of depths, and the cylinder of 4 mm diameter with nominal anechoic contrast properties was used for the quantitative measures described below.

The test phantom was imaged at room temperature through a layer of ultrasound gel (Litho-Clear, Sonotech Inc., Bellingham, Wash.) to represent the “no compression paddle” or “no mesh” condition, and it was also imaged through the gel and various test layers representing different types of compression paddles These test layers included a “1 mm mesh” (Textile Developments Associates, Inc. model PETKM3002, polyester, 1 mm×0.9 mm pore size, 0.23 mm thick, 34 g/m²) and a “2 mm mesh” (Textile Developments Associates, Inc. model PETKM3003, polyester, 2.0 mm×2.0 mm pore size, 0.15 mm thick, 14 g/m²). In addition, the 1 mm mesh was placed on top of a second 1 mm mesh for improved gel containment, the 2 mm mesh was placed on top of a 1 mm mesh for improved gel containment, a 3 mm spaced Dyneema (˜0.18 mm diameter) fish line weaved mesh, and a 2.5 mm thick solid plastic TPX (polymethylpentene) paddle were evaluated.

A linear matrix array ultrasound transducer (M12L, GE Healthcare, Milwaukee, Wis., USA) operating in a standard B-mode at a nominal center frequency of 10 MHz was translated across the surface of the phantom using a positioning system controlled by Labview software (National Instruments, Austin, Tex., USA). Neither image compounding, nor speckle reduction, nor frame averaging, nor beam steering was employed. The M12L transducer was connected to a GE Logiq 9 ultrasound unit, which was set for 100% acoustic output and eight focal zones starting at 1 cm depth, with a total imaging depth of 5 cm.

### Experimental Procedure

To test a given compression paddle candidate material, the transducer was initially raised from the phantom surface by two centimeters. The candidate material was then slid over the surface of the phantom and the transducer was lowered back to the original position (TPX excepted; see below). A bead of ultrasound gel was placed along the sweep-direction side of the transducer. The transducer was then test-swept once (90 mm sweep at 1 mm/second) across the phantom to spread the gel and confirm that coupling and imaging were satisfactory. The transducer was then swept back to the starting position. The following procedure was then carried out ten times in succession: A bead of gel was placed along the sweep-direction side of the transducer. The transducer was then swept (90 mm sweep at 1 mm/second) across the phantom with the GE Logiq 9 ultrasound system recording the ultrasound image cine loop. The long sweep ensured that the 4 mm cylinder was imaged from its most shallow visible position to the lowest depth of the ultrasound image. Post-sweep, the transducer was swept back to the starting position while the Logiq 9 image set was saved. The position of the candidate material on top of the phantom was then shifted slightly for the next run.

In the TPX case where there were no pores, the underside of the TPX plate was coupled to the phantom surface with a layer of gel.

### Analysis

### Determining Center Positions Through the Test Cylinders:

Cineloops (DICOM files) were opened on an analysis computer, automatically trimmed to the bounds of the scan region, and losslessly converted to TIFF stacks via in-house software developed in Matlab (TheMathworks, Inc. Natick, Mass.). An automatic region tracking algorithm, also developed in Matlab, was utilized to obtain the center positions of the 4 mm diameter “anechoic” cylinder region of interest (ROI) in each frame (of the sweep) of each cineloop.

### Calculating the Metrics for a Given Stack:

Signal amplitude for each pixel was log-decompressed from a log-linear grayscale map on the scanner:

Y=[20 log₁₀(X+1)]*255/DR, (Eq. 1)

where Y is the image value (i.e. values displayed in the image of the scanner after compression), X is the signal value, and DR is the dynamic range setting of the scanner (72 dB in all experiments). In the above relationship, the first half of the equation converts signal value to dB and the second half of the equation scales these dB values by the maximum gray-scale level (255) divided by the dynamic range (in dB). A high value of the dynamic range was chosen to accommodate the large differences in signal levels.

Next, three metrics were used to quantify image quality: signal strength, contrast, and contrast-to-noise ratio (CNR).

Signal strength was calculated as a function of depth from the images of the contrast detail phantom as follows. For each image at each depth, two rectangular regions of interest (ROIs) were defined as the background region. These ROIs, were positioned symmetrically on either side of the 4 mm diameter “anechoic” target cylinder, lying entirely within homogeneous background regions of the phantom. Their vertical positions were centered about the lesion center, and their height was slightly less than the diameter of the target. In each acquired image, the mean amplitude signal value of the background (BGMean) was estimated inside the uniform rectangle regions in the phantom at each depth to determine signal strength. The signal strength in dB was then defined as:

Signal_Strength_dB=20*log₁₀(BGMean) (Eq. 2).

The contrast, which quantifies how the signals from inside the target cylinder are perceptible against the background of the surrounding phantom background, was calculated as a function of depth. For each frame, at each depth, a circular ROI lying just within the 4 mm target and about the pre-determined target center was defined as the foreground region. The mean amplitude of the signal values within each circular ROI (TargetMean) was calculated at each depth. The mean amplitude of the background signal values (BGMean) at each depth was calculated in the region defined by the two rectangles, as was done previously. The final contrast was defined in dB as:

contrast_{dB}=20*log₁₀(TargetMean)−20*log₁₀(BGM)

Finally, the contrast-to-noise ratio (CNR) was estimated. It provides a gross imaging indicator of the perceptibility of details within the image which might be degraded by the noise. It was defined as the difference between mean signal values in the target cylinder and in the background, divided by the noise, which is the square root of the sum of the variances of the signal values in the target cylinder and background regions:

CNR=(TargetMean−BGM)/sqrt(σ²_{cyl}+σ²_{bg}),

where σ² denotes the variance over the region of interest.

The signal strength, contrast, and CNR values were determined over a depth range of 28 mm to 45 mm and were placed in 1 mm depth value bins for comparison. For contrast and signal strength values, mean bin values were compared as a ratio of the test scenario to the “no Mesh” case, then expressed in dB. For CNR values, mean bin values were compared as differences from the “no Mesh” case. Analyses were performed with JMP (SAS Institute, Microsoft Corporation, Cary, N.C.), and mean values over all depths were plotted for all cases. To show signal trends as a function of depth, key selected scenarios were plotted as well.

### RESULTS

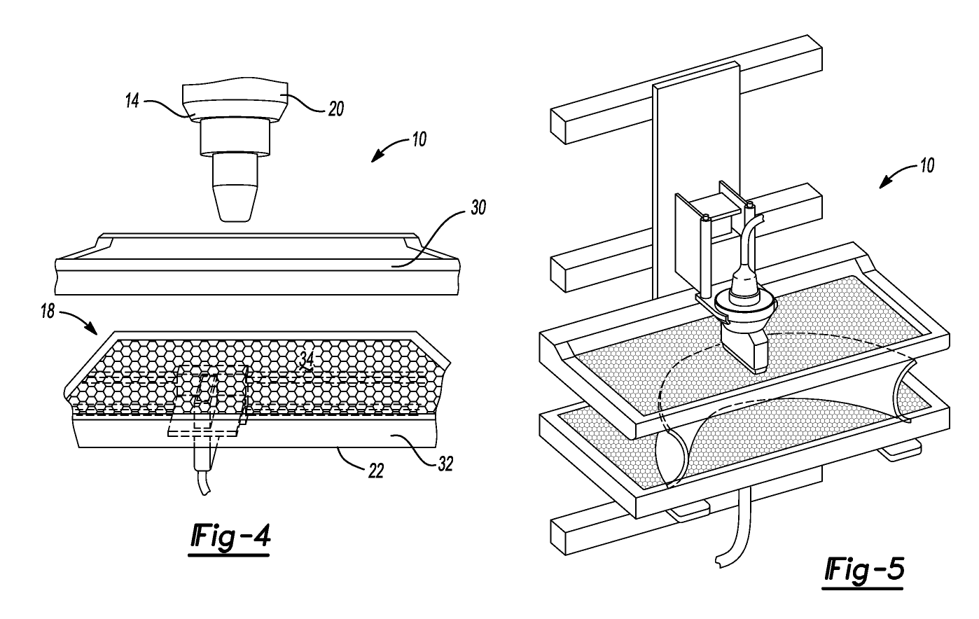

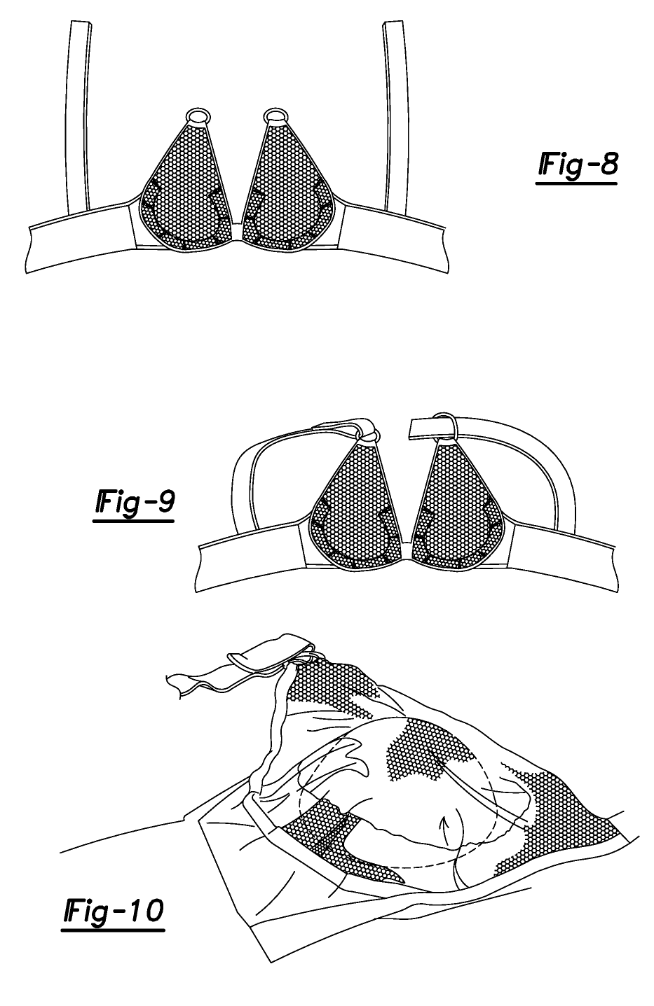

As mentioned in the Methods section, an ultrasound cross-sectional image of the test phantom alone is shown in FIG. 4. In contrast, FIG. 5 shows an ultrasound image of the same phantom acquired through the Dyneema mesh. Note the distinct surface artifacts due to the mesh. Also shown are the aforementioned nominally anechoic middle cylinder (4 mm) displayed on the right and the adjacent homogeneous background regions which were used for the quantitative measures reported below. Results for the seven interface scenarios (no mesh, 1 mm mesh, 2 mm mesh, 2 mm on 1 mm mesh, 1 mm on 1 mm mesh, 3 mm Dyneema, and TPX) are shown in FIGS. 6 through 8. Signal Strength and CNR are shown as a function of depth in FIGS. 9 and 10.

### Signal Strength

The relative mean background signal strength averaged over all depths is shown in FIG. 6. Each point represents mean values calculated over the ten sweeps and numerous depths within the 1 mm depth bin, i.e. nominally 150 values, and the vertical bars represent 95% confidence intervals of the data. Confidence intervals (95%) calculated over all depths indicated no significant difference between the scan with no overlying layers and the 2 mm mesh scenario for all calculated parameters. Additionally, there was no overlap of 95% confidence intervals between the TPX paddle scenario and any of the mesh cases. Performance of the Dyneema was somewhere in between as assessed by the signal strength and contrast indicators. More specifically, values for the “no mesh” and “2 mm mesh” cases basically overlap. The “1 mm mesh” and the “2 mm on 1 mm mesh” essentially overlap as well and display a slight decrease in signal strength from the “no mesh” and “2 mm mesh” cases, whereas the “1 mm on 1 mm mesh” and “3 mm Dyneema” scenarios display even lower values of signal strength. The signal strength through the TPX paddle was clearly lower than all other test scenarios, 4.6 dB below the “no mesh” scenario. FIG. 9 highlights the trend in signal strength as a function of depth as demonstrated by the “no mesh,” the “1 mm mesh,” and the “Dyneema” scenarios. These represent the range of the scenarios without introducing the confusion associated with too many plots in one figure. Signal strength generally decreases with depth as expected.

### Contrast and CNR

FIG. 7 and FIG. 8 graphically represent the contrast and contrast-to-noise ratio (CNR) values, respectively, obtained for the nominally anechoic 4 mm diameter targets. Contrast for the “no mesh” and “2 mm mesh” as well as the “1 mm mesh” and “2 mm on 1 mm mesh” scenarios display a trend similar to that in the case of the background signal strength. However, as evaluated by contrast, the quality of the images acquired through a 3 mm Dyneema mesh was overall worse than the quality of the images acquired through any of the polyester mesh fabrics, even the “1 mm on 1 mm” case, as shown by the 95% confidence intervals. The mean contrast level of the images acquired through TPX was again clearly lower than all of those acquired through any mesh by more than 2 dB, and more than 4 dB lower than the contrast measured with the best performing mesh, the “2 mm.” In the case of CNR values as shown in FIG. 8, distinctions among the mesh fabrics and the “no mesh” case are less pronounced. The CNR of the images acquired in the “3 mm Dyneema” case, however, was not obviously different from the CNR in the case of the TPX paddle. Nonetheless, FIG. 10 indicates that the “2 mm” and “1 mm mesh” display CNRs that are higher than the CNRs of the TPX paddle at all depths, and 95% confidence intervals have no overlap whatsoever in 15 of 18 depth bins calculated.

### SUMMARY AND CONCLUSION

The combined use of ultrasonography and mammography has been found to be superior to mammography alone in women with palpable masses or abnormal mammograms. However, separate examinations with images acquired in differing geometrical conditions confound the accuracy of localization. The mismatch rate was found to be at least 10% in one study consisting of women with predominantly fatty breasts and some with dense breasts, and it was expected to be much higher in the population of women with dense breasts and also in those with multi-centric disease. These problems could be mitigated with a system that could enable X-rays and ultrasound examinations to be taken with the patient in the same position and in the same sitting so that cross-modality registration is intrinsically achieved.

In addition, 3D imaging could potentially help to lower the mismatch. The automated or semiautomated acquisition of co-registered 3D dual-modality data sets is the ideal approach as it potentially alleviates the problem of operator dependence and provides greater assurance of localization of suspicious regions that could ultimately lead to improved characterization. Some studies have demonstrated that a combined 3D DBT and ABU co-registered imaging prototype system on an anthropomorphic breast phantom may enhance accurate and improved localization and characterization capabilities. A combined feature space of 3D X-ray and ultrasound attributes could be very valuable in characterizing regions of suspicion, as masses or simple cysts, benign or malignant, and in detecting masses that are occult with 2-D mammography, thereby leading to substantially improved breast imaging.

However, lack of realization of a dual-modality imaging system in which the breast is scanned through a compression paddle is most likely due to a combination of factors all leading to sub-optimal ultrasound image quality. These include highly attenuating compression paddles, substantial reverberations, image artifacts produced due to refraction through the paddle, and acoustic coupling difficulties on the sides of the breast and in the areolar region. Therefore, a better compression paddle is clearly needed in this system. A promising alternative compression paddle composition, polyester mesh, was tested in this study.

The 1 mm and 2 mm polyester mesh and combinations of these mesh fabrics were less attenuating and produced images with better CNR than the previously employed TPX plate. In contrast to another compression paddle choice, a Dyneema fish-line mesh, none of the polyester meshes produced noticeable artifacts in the images.

One possible issue with the use of 1 mm and 2 mm mesh in a compression paddle for X-ray breast imaging is the desirability to meet the Mammography Quality Standards Act (MQSA) requirement that the compression paddle “shall not deflect from parallel by more than 1.0 cm at any point on the surface of the compression paddle when compression is applied.” To satisfy this requirement, the mesh must be stretched very tightly across a stiff frame. We have developed two frames to accomplish this. One employs slots and splines much like those employed in a window screen and the other employs a ratcheting mechanism similar to that employed to tighten film in a 35 mm camera.

Use of a polyester mesh compression paddle instead of a solid plastic TPX compression paddle addresses some of the issues involved in making the automated ultrasound scanning part of a combined digital breast tomosynthesis/automated breast ultrasound system practical. In particular, mesh paddles reduce ultrasound attenuation and improve acoustic coupling. Other issues that are or will need to be addressed include development of ultrasound transducers that can be scanned over the paddle with their elements as close as possible to the chest wall to improve volume of coverage, scanning the breast from both sides (e.g., top and bottom) for improved image quality, and development of bubble-free gel delivery and containment methods to maintain an acoustic window at the periphery of the breast. Research on the latter two topics is underway in our laboratory. We will need the assistance of manufacturers in developing new transducers for this application.

### Alternatives

In some embodiments, alternative exist to improve the practicality of ultrasound scanning in the upright mammographic geometry for combined 3D X-ray and ultrasound breast imaging, dual sided breast imaging, and photoacoustic volume breast imaging. The methods include:

1) A polyester cloth gel roll;

2) Methods to push the gel roll against breast;

3) A device to raise the periphery of breast up to the compression paddle for improved breast to compression paddle contact;

4) A tilt compression paddle for improved compression paddle to breast contact;

5) Development of a gel that is compatible with both X-ray and ultrasound imaging; and

6) A rapid gel distribution system.

Generally, ultrasound scanning in the upright mammographic geometry will likely only be accepted as a clinical technique if it can be performed by a single technologist in a very efficient manner. The methods disclosed herein help achieve this goal.

### A Polyester Cloth Gel Roll.

For ultrasound scanning of the breast, it is critical that there be no air gaps between the breast and the ultrasound transducer. There are large gaps at the periphery of the breast which must be filled with ultrasound coupling media (gel). However, even the most viscous ultrasound gels are fairly free-flowing and will fall away from the breast when piled high next to the breast and will slide away from the breast due to gravity when imaging in oblique (e.g. Mediolateral oblique) and lateral projections. Placing the gel within a sack made of ultrasound transmissive material would keep the gel in one place. We have found that polyester cloth is excellent for this purpose. Sacks or rolls made of polyester cloth of the right thickness and weave keep most of the gel inside and allow a small amount to ooze out, which is ideal for acoustic coupling.

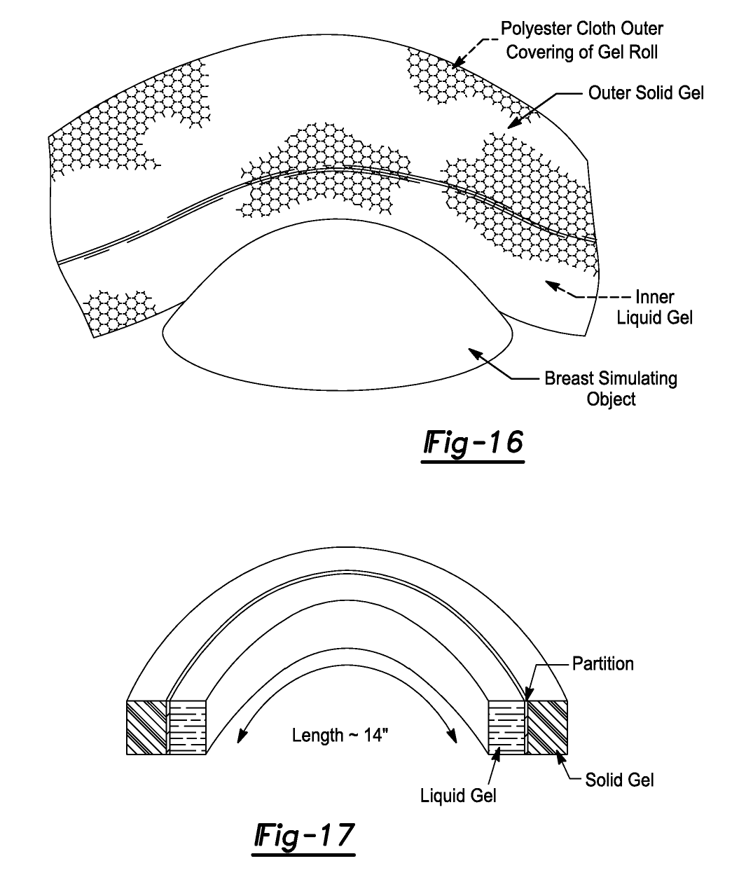

In one embodiment, as illustrated in FIG. 16, the roll is in the shape of a cylinder with a single seam. One of the ends is sealed and the roll is filled with gel after which that end is also sealed. In another embodiment, as illustrated in FIG. 17, a dual compartment roll is made of the cloth. A solid gel strip is placed in the back compartment of this roll and liquid gel is placed in the front compartment which comes in contact with the breast. There can be a cloth partition between the compartments, but this isn't required. The dual compartment roll may be more economical as it does not require the use of as much liquid gel and since the solid gel part does not come in contact with the patient, it can be cleaned and reused. Solid gels can be made of materials such as Versagel R750 (Calumet), ultrasound standoff pads (e.g. Parker Labs Aquaflex), Zerdine (CIRS, Inc.), and agarose.

### Methods to Push the Gel Roll Against Breast

For the coupling of the gel roll to the breast to be tight and free of air gaps and remain so throughout the ultrasound scanning procedure, some means must be used to push the gel roll against the breast and keep it there. Proposed methods include use of a belt that wraps around the gel roll and the back of the patient.

In one embodiment, as illustrated in FIG. 18, a concave shaped gel roll is used (e.g. the solid gel in the back of the roll is concave in shape) and the belt wraps around the roll, pushing the roll towards the breast throughout the contact area as the belt is tightened.

In another embodiment, as illustrated in FIG. 19, wedges are placed between the rectangular or cylindrically shaped gel roll so as to push the gel roll against the breast not only at the center (near the nipple) but also at the edges (e.g., at the axilla (near the arm pit)) for MLO projections. The belt could push against a rubber dam behind the gel roll or push directly against the gel roll. The rubber dam or gel roll could have belt loops through which the belt is threaded to maintain the belt's position. The belt could have a Velcro clasp or another type of clasp. The belt itself could be a self-adhering “ACE” type bandage.

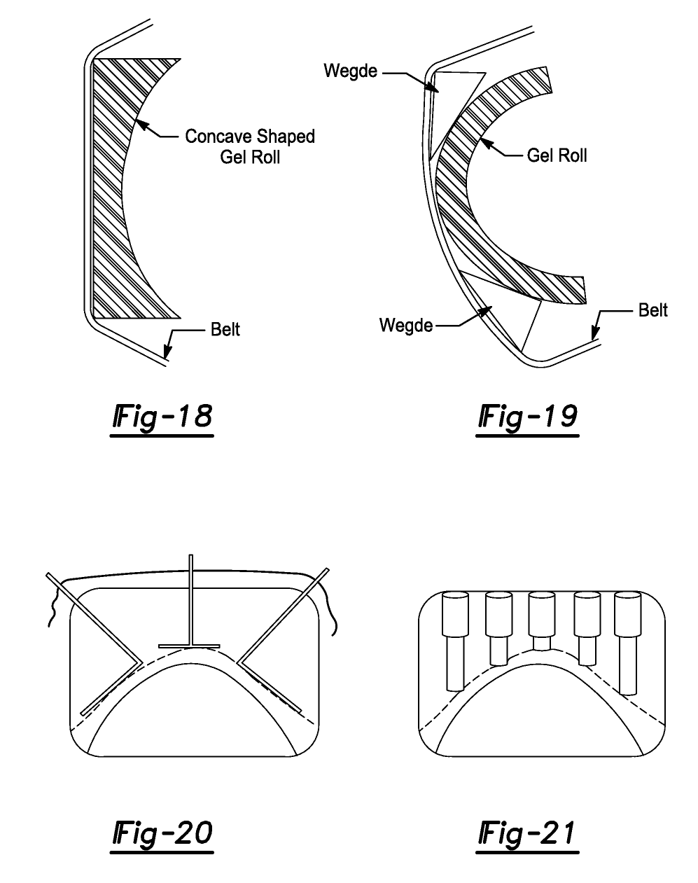

In a third embodiment, as illustrated in FIG. 20, a robotic mechanical device is employed (e.g. one that employs linear actuators.)

In a fourth embodiment, as illustrated in FIG. 21, two or more mechanical push rods can be employed. Either ratcheting mechanisms and/or screws are employed to lock the positions of the push rods. The push rods could be “L” or “T” shaped to apply pressure over extended areas. The push rods would be attached to a frame for locking their positions.

In a fifth embodiment, several rubber bladders are employed. These bladders push against the gel roll as the bladders inflate. The bladders could be placed within back restraining cups so the bladders push in a forward direction. The restraining cups would be attached to a frame. Several of the bladders could be tied together so the same hand pump could be used to inflate them the same amount.

### A Device to Raise the Periphery of Breast Up to the Compression Paddle for Improved Breast to Compression Paddle Contact.

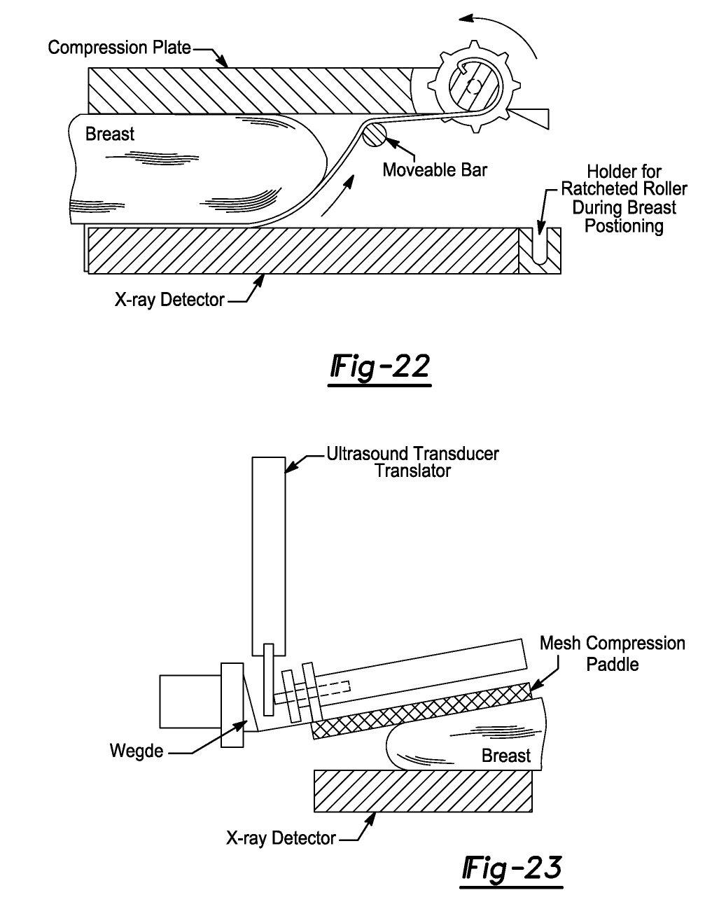

For single sided ultrasound scanning, as illustrated in FIG. 22, it can be helpful to bring the periphery of the breast up to the paddle so as to minimize the air gap at the periphery. A device similar to a hammock is provided. One method employs a device similar to a hammock to raise the outer edges of the breast up to the paddle for improved coupling.

One embodiment of this technique would use a square “shower-cap” made of elastic wrap that is wrapped on the X-ray detector during patient positioning. The elastic wrap would be stretched and wrapped (or clamped) around the detector sides to prevent wrinkles. The chest wall side of the wrap would be fixed at the detector edge by the patient's chest. After the breast was positioned and immobilized by compression, the anterior and two sides of the “shower cap” would be flipped up and wrapped (or clamped) around the sides of the compression paddle, then a bar would be raised up and attached to movable supports on the sides of the paddle that flip out of the way during breast positioning. The bar would be slid close to the breast to raise the wrap and breast as close as possible to the paddle. Finally, the bar supports would be clamped in position. Either a straight bar or a bar that is flexible and can be curved to roughly track the breast contour could be used. The latter produces breast-shape specific pivot points for the wrap and it should therefore increase the paddle-breast contact area compared to a straight bar.

In a second embodiment of this technique, as illustrated in FIG. 22, a thin elastic membrane would be attached to the chest wall edge of the breast support plate beneath the breast. The rest of the membrane would lie flat on the breast support plate while the technologist positions the breast. The anterior end of the membrane would be attached to a ratcheted roller mechanism which would be attached to the anterior side of the breast support plate. After positioning the patient's breast, the technologist would raise the ratcheted rolling mechanism up and attach it to a support at the anterior edge of the compression paddle. She would also attach a bar similar to the one discussed above. The moveable bar would be positioned close to the breast to act as a pivot for the membrane, resulting in improved lift. After the bar was positioned, the side supports for the bar would be clamped in place. The technologist would then rotate the roller, tightening the membrane (much like installing a roll of film in a 35 mm camera). This in turn would raise the breast up to the mesh paddle where it would be in contact with ultrasound gel.

Side view illustrating “hammock” device for moving outer edge of breast up to the compression paddle to increase area of coverage in the ultrasound scans through the paddle. The membrane shown in blue is wrapped around a ratcheted roller mechanism which is turned to move the membrane and breast upward. A movable bar acts as a pivot point for the membrane.

Different elastic wraps or membranes could be used. For sterility reasons a new wrap or membrane would be employed for each patient. The ideal material would be thin and flexible, have low X-ray attenuation, be wrinkle and tear resistant, and be hypoallergenic, strong and relatively inexpensive. A wrap or membrane that is sticky might also be desirable as it would help prevent the breast from moving.

### A Tilt Compression Paddle for Improved Paddle Breast Contact.

Another method that would significantly increase the area of the breast that is in direct contact with the compression paddle and thereby minimize the air gap area at the periphery of the breast for ultrasound scanning is to use a tilt compression paddle as illustrated in FIG. 23. Such paddles are employed to push the breast tissue near the nipple closer to the X-ray detector in conventional mammography for improved spatial resolution in this region of the X-ray image. It would have a different purpose for this application, as it would put the mesh paddle closer to the top surface of the breast for a smaller air gap at the periphery for ultrasound scanning. Another difference is that this application employs a mesh paddle whereas conventional mammography employs a solid plastic (e.g. polycarbonate) paddle. The paddle could be tilted by utilizing a wedge where the paddle connects to the compression device. Other means including variable tilt could be achieved by mechanical means (e.g. using one or more hinges, frames and locking screws).

Development of a Gel that is Compatible with Both X-Ray and Ultrasound Imaging.

The entire task of performing combined 3D X-ray (tomosynthesis) and 3D automated ultrasound scanning could be simplified if the X-ray imaging could be performed while the ultrasound scanning gel is in place. This would minimize the time between the two imaging modes and thereby minimize potential patient movement between the two modality acquisitions, which could be deleterious to the co-location of lesions found with the two modalities. The entire procedure could be performed more quickly.

Unfortunately, the water based gels that are employed in ultrasound imaging are very attenuating for X-rays and would degrade X-ray image quality. A compromise would be a gel that has low X-ray and ultrasound attenuation (absorption), a speed of sound that is similar to that of breast tissue, a fairly high viscosity and is bubble-free. A gel that has a high lipid (fat) content would meet this requirement. Unfortunately, most high fat gels (e.g. petrolatum) are very difficult to clean off of skin and are difficult to make bubble-free. One that is promising is Versagel 500 by Calumet, Inc. Methods to make it less sticky and easier to cleanup include the addition of surfactants and/or emollients. Another promising gel is mayonnaise and egg-free mayonnaise. Unfortunately, removal of air bubbles has been a problem for the mayonnaise gels.

### Rapid Bubble-Free Gel Distribution

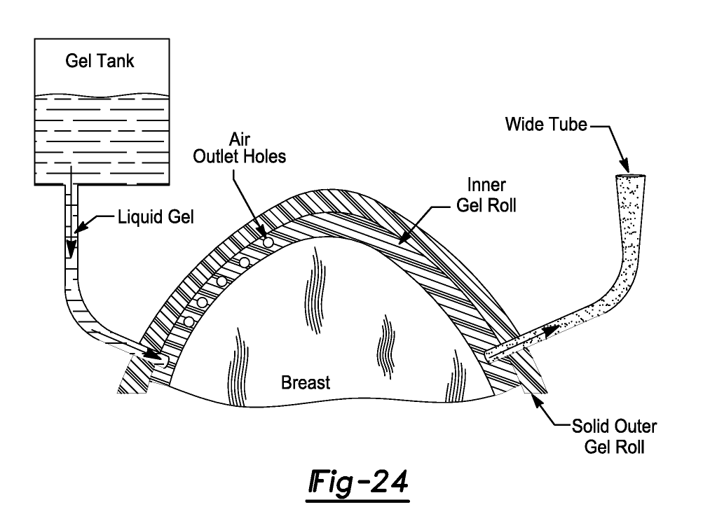

Rather than placing a filled gel roll next to the breast, which could result in air bubbles at the interface, a technique that rapidly fills the gel roll while the gel roll is in contact with the breast could be employed as illustrated in FIG. 24. In an embodiment of this device, a large tank containing gel would be connected to the gel roll via a wide tube on one side of the gel roll. Gel would flow through the tube via a gravity feed. The gel roll would have outlet holes on the top through which air would flow as the gel roll was being filled. At the other end of the gel roll there would be a second tube through which the gel would exit.

The foregoing description of the embodiments has been provided for purposes of illustration and description. It is not intended to be exhaustive or to limit the disclosure. Individual elements or features of a particular embodiment are generally not limited to that particular embodiment, but, where applicable, are interchangeable and can be used in a selected embodiment, even if not specifically shown or described. The same may also be varied in many ways. Such variations are not to be regarded as a departure from the disclosure, and all such modifications are intended to be included within the scope of the disclosure.

What is claimed is:

1. A system for imaging comprising:
a first scanning system scanning a portion of a body, the first scanning system employing at least ultrasonic scanning;
a compression system applying a compressing force to the portion of the body being scanned; and
a device for containing an acoustic coupling gel for enhanced acoustic coupling with the portion of body.

2. The system according to claim 1 wherein the compression system applies the compressing force to the portion of the body while the first scanning system simultaneously scans the portion of the body.

3. The system according to claim 1 wherein the first scanning system comprises:
a first ultrasonic transducer outputting an ultrasonic signal to a first side of the portion of the body; and
a second ultrasonic transducer outputting an ultrasonic signal to a second side of the portion of the body, the second side of the portion of the body being opposite of the first side of the portion of the body,
wherein the first ultrasonic transducer is operable simultaneously with the second ultrasonic transducer.

4. The system according to claim 1, further comprising:
a second scanning system scanning the portion of the body, the second scanning system employing X-ray scanning.

5. The system according to claim 1, further comprising:
a second scanning system scanning the portion of the body, the second scanning system employing digital breast tomosynthesis (DBT).

6. The system according to claim 1 wherein the compression system compromises a frame portion having a mesh extending there across.

7. The system according to claim 6 wherein the mesh permits a portion of the acoustic coupling gel to pass therethrough.

8. The system according to claim 6 wherein the mesh is substantially transparent during digital breast tomosynthesis (DBT) imaging, mammography imaging, and ultrasound (US) imaging.

9. The system according to claim 1 further comprising:
a force feedback system outputting a signal to an operator indicative of the compressing force applied to the portion of the body.

10. The system according to claim 9 wherein the force feedback system outputs a tactile signal to the operator exerting a force on the operator.

11. The system according to claim 9 wherein the force feedback system comprises a joystick.

12. A method of imaging a portion of a body, the method comprising:
applying a first compressing force to the portion of the body;
physically constraining an acoustic coupling gel against the portion of the body; and
ultrasonically imaging the portion of the body using an ultrasonic transducer through the acoustic coupling gel while simultaneously performing the steps of applying a first compressing force to the portion of the body.

13. The method according to claim 12, further comprising:
imaging the portion of the body using an X-ray imager while simultaneously performing the steps of applying a first compressing force to the portion of the body.

14. The method according to claim 12, further comprising:
imaging the portion of the body using an digital breast tomosynthesis (DBT) system while simultaneously performing the steps of applying a first compressing force to the portion of the body.

15. The method according to claim 12 wherein the physically constraining an acoustic gel comprising employing a gel dam to retain the acoustic coupling gel against the portion of the body.

16. The method according to claim 12 wherein the physically constraining an acoustic gel comprising employing a bra made of a mesh material to retain the acoustic coupling gel against the portion of the body, the mesh material being sufficient porous to permit the acoustic coupling gel to flow partially therethrough.

17. The method according to claim 12 wherein the ultrasonically imaging the portion of the body comprises ultrasonically imaging opposing side of the portion of the body simultaneously using the ultrasonic transducer and a second ultrasonic transducer.

18. The method according to claim 12 further comprising:
applying a second compressing force supplementing the first compressing force, the second compressing force being applied by the ultrasonic transducer.

19. The method according to claim 12 wherein the applying a first compressing force is applied automatically by a compression system.

20. The method according to claim 19 wherein the applying a first compressing force is applied automatically by a compression system and a force feedback indicative of the compressing force is outputted to an operator.
